(19) United States
(12) Patent Application Publication
(10) Pub. No.: US 2009/0196923 A1
(43) Pub. Date: Aug. 6, 2009

(54) CONTROLLED RELEASE FORMULATION COMPRISING ANTI-EPILEPTIC DRUGS

(76) Inventors: Jayanta Kumar Mandal, Gujarat (IN); Nitesh Nalinchandra Pandya, Gujarat (IN); Sumitra Ashok Pillai, Gujarat (IN); Kirti Bansidhar Maheshwari, Gujarat (IN)

Correspondence Address: HAMRE, SCHUMANN, MUELLER & LARSON, P.C., P.O. BOX 2902, MINNEAPOLIS, MN, 55402-0902, US

(21) Appl. No.: 12/303,021

(22) PCT Filed: Apr. 23, 2007
(86) PCT No.: PCT/IN07/00160

§ 371 Date: Feb. 25, 2009

(30) Foreign Application Priority Data
Apr. 26, 2006 (IN) 651/MUM/2006

(51) Int. Cl. A61K 9/22; A61P 25/08

(52) U.S. Cl. 424/468

(57) ABSTRACT

The present invention relates to pharmaceutical formulation of antiepileptic drug preferably oxcarbazepine. The formulation comprises multiple tablets or pellets of immediate release or controlled release nature, which are filled, inside the capsule to provides drug effect for 24 hours and is suitable for once a day administration. The patent also provides process of preparation of the dosage form.

### FIELD OF INVENTION

The present invention relates to a pharmaceutical formulation of antiepileptic drug preferably oxcarbazepine comprising multiple tablets or pellets filled inside capsule wherein the individual tablet or pellet are either immediate release or controlled release in nature.

This formulation can be used once a day or twice a day, preferably once a day.

This formulation maintains a constant drug plasma level for 24 hours and hence reduces fluctuation in blood level caused by repeated dosing of immediate release drug. The invention further describes the method of preparation of the said pharmaceutical formulation.

### BACKGROUND OF THE INVENTION

Epileptic seizures are mainly of two types: partial seizures and generalized seizures. Partial seizures can again be of three type; i.e. simple partial, complex partial and partial with secondarily generalized tonic clonic seizure. Generalized seizures are classified as absence seizure, myoclonic seizure and tonic-clonic seizure.

Based on their mechanism of action, anti-epileptic drugs can be classified as sodium channel blockers, calcium current inhibitors, gamma-aminobutyric acid (GABA) enhancers, glutamate blockers, carbonic anhydrase inhibitors etc.

Oxcarbazepine is keto analog of carbamazepine and is an important antiepileptic drug, which acts by sodium channel blocker mechanism. Chemically oxcarbazepine is known as 10, 11-dihydro-10-oxocarbamazepine.

Oxcarbazepine gets converted to its active metabolite 10-monohydroxy derivative which is responsible for the pharmacological effect. Oxcarbazepine is considered for monotherapy or adjunct therapy for partial seizures. It is also considered for first line therapy in the treatment of epileptic seizures in many countries.

Oxcarbazepine has poor water solubility. This causes problem in formulating the desired dosage form. In addition of being insoluble or having low solubility, it is also difficult to achieve a high loading dose of oxcarbazepine in multi tablets or pellets when formulated as sustained release dosage forms. The term high loading as used in this application shall mean at least twenty percent (20%) Oxcarbazepine by weight of total dose of Oxcarbazepine.

So to prepare a dosage form having controlled release profile leading to enhance bioavailability of oxcarbazepine is the aim of this invention.

Repeated dosing of immediate release drug causes fluctuation of drug concentration level in blood. So to prepare a formulation, which can preferably be administered once a day and which reduce the drawback of repeated dosing is also an aim of this invention.

A pharmaceutical dosage form which can impart both immediate drug effect and sustained drug effect to a patient suffering from epileptic seizures and which can be suitable for once a day use providing patient compliance is a further aim of this invention.

### PRIOR ART

WO9835681 discloses a film coated oxcarbazepine formulation wherein the median particle size of the active ingredient is approximately 2 to 12 μm, preferably 4 to 12 μm.

Particle size reduction is time consuming and tedious process. Difficulty in handling and increased chance of drug loss during the process are the further drawbacks of micronized particles.

U.S. Pat. No. 5,472,714 reveals a double-layered oxcarbazepine tablet containing an inner hydrophilic permeable layer containing white pigment and an outer hydrophilic permeable layer containing white pigment in combination with iron (II) oxide pigment.

WO2002094774 describes a composition for oral administration comprising oxcarbazepine and a wetting agent.

WO2004026314 describes once a day oral dosage form consisting of a tablet core and a coating wherein the dosage form produces a constant monohydroxy derivative of oxcarbazepine plasma level for 24 hours for the treatment of epilepsy.

US20040142033 describes a pharmaceutical composition containing oxcarbazepine with sustained release of the active ingredient. The sustained release effect is obtained through rate controlling polymers used along with the active pharmaceutical ingredient in the formulation core.

The above prior arts have somehow abridged the problem associated with oxcarbazepine formulation, but there still remains a pressing need for improvement in the existing formulations and to prepare a dosage form which can crack the adversities related to the active ingredient oxcarbazepine and which can be well-suited to an epileptic patient for once a day use.

### OBJECT OF THE INVENTION

The instant object of the invention is to provide a pharmaceutical dosage formulation for epileptic patients, which is controlled release in nature and which imparts a sustained drug effect.

Another object of the invention is to provide a pharmaceutical preparation which can be used once a day or twice a day, preferably once a day by a patient suffering from epileptic seizure.

The next object of the invention is to prepare a pharmaceutical formulation of antiepileptic drug which reduces the fluctuation of blood level caused by repeated dosing of immediate release tablet.

Another object of the invention is to provide a pharmaceutical preparation preferably capsule comprising of multi-tablets or pellets which individually releases the active ingredient in immediate and/or in sustained duration of time providing an overall sustained drug action.

Further one more object of the invention is to prepare a pharmaceutical formulation comprising antiepileptic drug preferably carbamazepine derivative, more preferably oxcarbazepine.

Still one more object of the invention is to provide a process of preparation of orally administrable sustained release oxcarbazepine formulation.

### SUMMARY OF THE INVENTION

The present invention provides a pharmaceutical composition(s) and/or dosage form(s) for oral administration suitable for once a day or twice a day preferably once a day to a patient suffering from epileptic seizure, said composition comprising multi tablets or pellets in capsule wherein the individual tablets or pellets are of immediate release or controlled release nature. This formulation reduces fluctuation in blood level caused by repeated dosing of immediate release dosage form.

### DETAIL DESCRIPTION OF THE INVENTION

The pharmaceutical formulation of the present invention comprises multiple units, which are tablets or pellets in a capsule. The tablets or pellets filled inside the capsule are of immediate release and/or controlled release nature.

The terms controlled release, sustained release, sustained action, prolonged action, extended action; extended release, timed release, pulsatile release etc. should be considered to be within the scope of the present invention.

The term unit used in the description means individual tablet or pellet of immediate release or controlled release nature.

The immediate release tablets or pellets provides an instant drug effect which is then maintained for prolonged time duration by the remaining controlled release tablets or pellets and hence the formulation of the present invention provides an overall sustained drug action.

In the formulation of the present invention one or more than one tablet are of immediate release and rest are of controlled release nature. Similarly in formulation comprising pellets, amount of controlled release pellets are total pellets minus amount of pellets sufficient to bring immediate drug effect.

A part of the formulation providing immediate drug release comprises uncoated or coated tablets or pellets, whereas the rest part of the formulation providing controlled drug release are coated by rate controlling polymer coating solution which controls the drug release in response to the pH environment at a given site in the distal region of the gastro intestinal tract.

Drug release from controlled release units of the dosage form at different sites of the distal region of gastro intestinal tract depends upon use of different pH dependent release controlling polymers in the controlled release coating solution. The same rate control polymer containing coating solution when used in different proportion to coat the different units of the dosage form may also bring controlled drug release to provide an overall 24 hours drug effect of the dosage form.

Weight of each individual tablet in the formulation of present invention is 5-50% of the total weight of formulation and in case of pellets each pellet can be from 0.1 to 5% of total weight of the formulation.

The immediate release tablets comprises core tablet optionally coated with film coating solution. The controlled release tablets comprise film coated immediate release tablets with additional coating of controlled release coating solution.

The immediate release pellets comprises core pellets optionally coated with film coating solution. The controlled release pellets comprises film coated immediate release pellets with additional coating of controlled release coating solution.

Core of the immediate release and sustained release tablet(s) of the present invention comprises of 10-90% of the active pharmaceutical ingredient, 1-15% of the binder, 1-15% of the disintegrant, 0.25-10% of the lubricant and a solvent system as per the requirement.

Core of the immediate release and sustained release pellet(s) comprises of 10-90% of the active ingredient and 1-15% of formulating excipients comprising 1-15% diluent, 1-20% binder and 1-4% of lubricant to the weight of the core pellet.

The amount of film coating around the core tablet and pellet is calculated as the percentage weight gain over the core tablet or pellet. In the formulation of the present invention, the percentage weight gain over the core tablet by the film coating solution is 0.5 to 5%. The preferred percentage weight gain over the core tablet or pellet by the film coating solution is 1.5 to 3.5% and most preferably between 2 to 3%.

The controlled release coating around the immediate release film coated tablet or pellet is calculated as the percentage weight gain over the immediate release tablet or pellet.

In the formulation of the present invention, the controlled release units which release the drug at pH 5 to 6 of the gastro intestinal tract, the percentage weight gain by the controlled release coating solution over the immediate release unit is 5 to 25% of the weight of the film coated immediate release unit, more preferably between 8 to 16%.

In the controlled release units from which drug release occurs at pH 6 to 8 of the gastro intestinal tract, the percentage weight gain by the control release coating solution over the immediate release unit is 5 to 50% of the weight of the film coated immediate release unit, more preferably between 15 to 35%.

The amount of controlled release coating solution used to coat the film coated immediate release pellets is 5 to 25% weight gain over the film coated immediate release pellets.

The film coating solution comprises coating agent preferably coating polymers suitable to prevent the contact between core and control release coating layer. Of the total percentage weight gain of film coating, 1-20% comprises film coating polymer, 0.1-15% comprises plasticizer, optionally 0.2-15% comprises acid neutralizer, along with a pharmaceutically acceptable and suitable solvent.

The controlled release units of the dosage form from which drug release occurs at pH 5 to 6 of the gastro intestinal tract comprises controlled release coating layer over the film coated immediate release units. Of the total percentage weight gain of this controlled release coating layer over the film coated immediate release unit, 5-50% comprises controlled release polymers or retardants, 0.1-10% comprises plasticizer, 0.01-15% of emulsifier, 0.05-15% of antisettling agents, 0.01-15% of pigments, optionally with 0.2-10% acid neutralizer, along with a pharmaceutically acceptable and suitable solvent.

The controlled release units of the dosage form from which drug release occurs at pH 6 or 8 of the gastro intestinal tract, comprises controlled release coating layer over the film coated immediate release units. Of the total percentage weight gain of this controlled release coating layer over the film coated immediate release unit, 5-50% comprises controlled release polymers or retardants, 0.1-20% comprises plasticizer, 0.05-15% of antisettling agents, 0.01-15% of pigments, 0.5-15% of acid neutralizer, along with a pharmaceutically acceptable and suitable solvent.

The active pharmaceutical ingredient in the formulation of the present invention is selected from anti-epileptic drugs preferably carbamazepine or its derivative, more preferably oxcarbazepine or pharmaceutically acceptable salt thereof. The amount of active pharmaceutical ingredient in the formulation of present invention is 150 to 900 mg.

The preferred binder used in the formulation of the present invention is selected from the group comprising of but not limited to starch, cellulose and cellulose derivatives, polyvinylpyrollidone, ethyl cellulose, polyethylene glycol, polyvinyl alcohol, and the like.

The preferred lubricant used in the formulation of the present invention is selected from the group comprising of but not limited to magnesium stearate, other alkali earth metal stearate; colloidal anhydrous silica, talc, sodium stearyl fumarate, glyceryl monostearate and the like.

The preferred disintegrant used in the formulation of present invention is selected from the group comprising of but not limited to cross-linked polymers such as crospovidone; starch or modified starch such as sodium starch glycolate, clays such as bentonite or veegum; celluloses or cellulose derivatives; crosslinked cellulose such as croscarmellose sodium, resins such as polacrillin potassium and the like.

The coated tablets or pellets in the formulation of the present invention comprises coating polymer, which can be selected from water-soluble or water insoluble film coating or release controlling polymer. The controlled release tablets, or pellets may be coated with a rate controlling polymer coating or a combination of hydrophilic film coating & a rate controlling polymeric coating.

The preferred polymers for film coating of the tablet core or pellet core are selected from the group comprising of but not limited to zein, cellulose and cellulose derivative like hydroxypropylmethylcellulose, hydroxyethyl cellulose, methyl cellulose, hydroxypropylcellulose; polyvinylpyrollidone, polyvinyl alcohol and the like.

The preferred coating polymers for controlled release coating of the immediate release film coated tablet or pellet is selected from the group comprising of but not limited to methacrylic acid copolymers preferably methacrylic acid copolymer type-A, methacrylic acid copolymer type-B, methacrylic acid copolymer type-C, polyvinyl acetate phthalate, hydroxypropylmethylcellulose phthalate, cellulose acetate phthalate and the like.

The preferred acid neutralizer used in the formulation of present invention is selected from the group comprising of but not limited to magnesium oxide, sodium hydroxide, potassium hydroxide, ammonia, solution, ammonium chloride and the like.

The preferred plasticizer used in the formulation of present invention is selected from the group comprising of but not limited to polyethylene glycol, propylene glycol, diethyl phthalate, triethyl citrate, acetylated triethyl citrate, methyl citrate, triacetin and the like.

The preferred anti settling agents used in the formulation of present invention is selected from the group comprising of but not limited to talc, anhydrous silica and the like.

The preferred pigments used in the formulation of present invention is selected from the group comprising of but not limited to iron oxide, titanium dioxide, phthalocyanine blue and the like.

The preferred emulsifier used in the formulation of present invention is selected from the group comprising of but not limited to tween-80, lecithin, a polysorbate, polyoxyethylene hardened castor oil, macrogol and the like.

The preferred capsule used to formulate the dosage form of the present invention is selected from but not limited to hard gelatin capsule, soft gelatin capsule, HPMC capsules and the like.

### Process for Preparation of the Dosage Form:

The dosage form of the present invention is formulated by preparing immediate release and controlled release tablets or pellets and filling them in a capsule wherein the dosage form is suitable for once a day administration.

### Preparation of Immediate Release Tablet(S):

The API and excipients like diluent and disintegrant are granulated with binder preparation, which is then dried and lubricated. Then it is compressed to form tablets. The immediate release tablets may remain uncoated or coated with a film coating solution.

### Preparation of Controlled Release Tablet(S):

The uncoated or film coated core tablets are coated by rate controlling coating solution to control the drug release in response to the pH environment at a given site in the distal region of the gastro intestinal tract.

### Preparation of Immediate Release and Controlled Release Pellet(S):

Wet mass of drug & binder solution are extruded, spheronised and dried to obtain dried pellets. The immediate release pellets are either uncoated or coated with the film coating solution. The controlled release pellets are film-coated pellets additionally coated with a rate controlling coating solution.

Throughout this specification it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

### EXAMPLE

The present invention has been described by way of example only, and it is to be recognized that modifications thereto falling within the scope and spirit of this specification, and which would be obvious to a person skilled in the art based upon the disclosure herein, are also considered to be included within the scope of this invention.

The formulation of the present invention is described through examples comprising multiple tablets or pellets of oxcarbazepine contained inside a hard gelatin capsule.

The invention is illustrated by the following non-limiting example(s). These are for illustration purpose only and should not be construed as limiting the scope of the invention.

### Example-1

The dosage form is capsule formulation prepared by encapsulating a combination of immediate and sustained release tablets.

Tablets comprises core which remain uncoated or film coated for immediate release of drug and coated with controlled release coating solution for sustained or controlled release of drug providing an overall 24 hours drug effect of the dosage form. Common tablet core was used to prepare immediate and controlled release units.

TABLE 1
Composition for common core tablet

| | | |
|---|---|---|
| Sr. No. | Ingredients | Percentage |

Dry Mixing

| | | |
|---|---|---|
| 1 | Oxcarbazepine | 86.20% |
| 2 | Crospovidone | 9.20% |

Granulation

| | | |
|---|---|---|
| 3 | PVP K 30 | 2.87% |
| 4 | Water | — |

Lubrication

| | | |
|---|---|---|
| 5 | Colloidal Anhydrous Silica | 0.87% |
| 6 | Magnesium Stearate | 0.87% |

### Procedure:

Weighed quantity of the active ingredient & disintegrant was granulated with the binder preparation, sieved and lubricated using lubricants. The prepared blend was compressed into tablet using a rotary compression machine. For immediate drug release, the core tablets remain uncoated or coated with film coating solution of table-2. The film-coated tablets were further coated with controlled release coating solution of table 3 or 4 for control release of the drug.

TABLE 2
Composition for Film coating:

| Sr. No. | Ingredients | Percentage |
|---|---|---|
| 1 | HPMC E5 | 1.4% |
| 2 | Triethyl citrate | 0.41% |
| 3 | Light Magnesium Oxide | 0.14% |
| 4 | Water | Q.S. |

TABLE 3
Composition for rate controlling polymer coating, which facilitates
drug release at pH 5 to 6 of the gastro intestinal tract.

| Sr. No. | Ingredients | Percentage |
|---|---|---|
| 1 | Eudragit L30 D55 | 8.91% |
| 2 | Sodium Hydroxide pellets | 0.23% |
| 3 | Diethyl Phthalate | 0.38% |
| 4 | Tween-80 | 0.04% |
| 5 | Ferric Oxide Red | 0.13% |
| 6 | Colloidal Anhydrous Silica | 0.31% |
| 7 | Water | Q.S. |

TABLE 4
Composition for rate controlling polymer coating, which facilitates
drug release at pH 6 to 8 of the gastro intestinal tract.

| Sr. No. | Ingredients | Percentage |
|---|---|---|
| 1 | Eudragit S100 | 11.24% |
| 2 | Ammonia Solution (1 N) | 6.18% |
| 3 | Triethyl Citrate | 2.77% |
| 4 | Ferric Oxide Yellow | 0.75% |
| 5 | Titanium Dioxide | 0.75% |
| 6 | Talc | 3.22% |
| 7 | Water | Q.S. |

### Example-2

The dosage form is capsule formulation prepared by encapsulating a combination of immediate and sustained release tablets.

Tablets comprises core which remains uncoated or film coated for immediate release of drug and coated with controlled release coating for sustained release of drug providing an overall 24 hours drug effect of the dosage form. Tablet core used for immediate release and controlled releases units were different.

### Preparation of Immediate Release Units:

TABLE 5
Composition of immediate release core tablet

| | | |
|---|---|---|
| Sr. No. | Ingredients | Percentage |

Dry Mixing

| | | |
|---|---|---|
| 1 | Oxcarbazepine | 85.23% |
| 2 | Crospovidone | 5.68% |

Granulation

| | | |
|---|---|---|
| 2 | PVP K 30 | 2.87% |
| 3 | Water | — |

Extra granular Ingredients

| | | |
|---|---|---|
| 4 | Crospovidone | 4.54% |

Lubrication

| | | |
|---|---|---|
| 5 | Colloidal Anhydrous Silica | 0.85% |
| 6 | Magnesium Stearate | 0.85% |

### Procedure:

Weighed quantity of the active ingredient and disintegrant was granulated with the binder preparation, sieved and lubricated with lubricants. The prepared blend was compressed into tablet using a rotary compression machine. For immediate drug release, the core tablets remain uncoated or were coated with film coating solution of table-6.

TABLE 6
Composition for Film coating:

| Sr. No. | Ingredients | Percentage |
|---|---|---|
| 1 | HPMC E5 | 1.4% |
| 2 | Talc | 0.41% |
| 3 | PEG 4000 | 0.14% |
| 4 | Water | Q.S. |

### Preparation of Controlled Release Units Releasing Drug at pH 5 to 6 of the Gastro Intestinal Tract:

TABLE 7
Composition of controlled release core tablet from which drug
release occurs at pH 5 to 6 of the gastro intestinal tract.

| | | |
|---|---|---|
| Sr. No. | Ingredients | Percentage |

Dry Mixing

| | | |
|---|---|---|
| 1 | Oxcarbazepine | 88.24% |
| 2 | Crospovidone | 7.00% |

Granulation

| | | |
|---|---|---|
| 3 | PVP K 30 | 2.94% |
| 4 | Water | — |

Lubrication

| | | |
|---|---|---|
| 5 | Colloidal Anhydrous Silica | 0.91% |
| 6 | Magnesium Stearate | 0.91% |

### Procedure:

Weighed quantity of the active ingredient and disintegrant was granulated with the binder preparation, sieved and lubricated with lubricants. The prepared blend was compressed into tablet using a rotary compression machine. The core tablets were coated with film coating solution of table-2 or 6 and further coated with controlled release coating solution of table 8.

TABLE 8
Composition of rate controlling polymer coating which imparts
drug release at pH 5 to 6 of the gastro intestinal tract.

| Sr. No. | Ingredients | Percentage |
|---|---|---|
| 1 | Eudragit L30 D55 | 9.29% |
| 2 | Triethyl citrate | 0.27% |
| 3 | Titanium dioxide | 0.31% |
| 4 | Ferric Oxide Red | 0.13% |
| 5 | Water | Q.S. |

### Preparation of Controlled Release Units Releasing Drug at pH 6 or 8 of the Gastro Intestinal Tract:

TABLE 9
Composition of controlled release core tablet from which drug
release occurs at pH 6 to 8 of the gastro intestinal tract.

| | | |
|---|---|---|
| Sr. No. | Ingredients | Percentage |

Dry Mixing

| | | |
|---|---|---|
| 1 | Oxcarbazepine | 86.20% |

Granulation

| | | |
|---|---|---|
| 2 | HPMC K4M | 9.20% |
| 3 | PVP K 30 | 2.87% |
| 4 | Water | — |

Lubrication

| | | |
|---|---|---|
| 5 | Colloidal Anhydrous Silica | 0.86% |
| 6 | Magnesium Stearate | 0.86% |

### Procedure:

Weighed quantity of the active ingredient was granulated with the binder preparation, sieved and lubricated with lubricants. The prepared blend was compressed into tablet using a rotary compression machine. The core tablets were coated with film coating solution of table-2 or 6 and further coated with controlled release coating solution of table 10.

TABLE 10
Composition of rate controlling polymer coating, which imparts
drug release at pH 6 to 8 of the gastro intestinal tract.

| Sr. No. | Ingredients | Percentage |
|---|---|---|
| 1 | Eudragit S100 | 12.06% |
| 2 | Ammonia Solution (1 N) | 6.63% |
| 3 | Triethyl Citrate | 2.98% |
| 4 | Ferric Oxide Yellow | 0.81% |
| 5 | Titanium Dioxide | 0.81% |
| 6 | Talc | 3.45% |
| 7 | Water | Q.S. |

### Dissolution of Controlled Release Capsule Dosage Form Comprising Immediate Release and Controlled Release Tablets:

The dissolution profile of the formulation of the present invention is as below:

TABLE 11
Dissolution Profile:

| Sr. No. | Time (Hours) | Dissolution Medium | Limits |
|---|---|---|---|
| 1 | 3 | 0.1 N HCl | 5%-25% |
| 2 | 6 | Trisodium buffer pH 5.5 | 25%-60% |
| 3 | 9 | Trisodium buffer pH 6.8 | 40%-70% |
| 4 | 12 | Trisodium buffer pH 6.8 | 50%-80% |
| 5 | 18 | Trisodium buffer pH 6.8 | NLT 70% |

### Example-3

The dosage form is capsule formulation prepared by encapsulating a combination of immediate and sustained release pellets.

Pellets comprises core which remain uncoated or film coated for immediate release of drug and coated with controlled release coating for sustained release of drug providing an overall 24 hours drug effect of the dosage form. Common pellet core was used for preparing immediate and controlled release units.

TABLE 12
Composition for common core pellets:

| | | |
|---|---|---|
| Sr. No | Ingredient | Qty (%) |

Granulation

| | | |
|---|---|---|
| 1. | Oxcarbazepine | 10-90% |
| 2. | HPMC E5 | 1-20% |
| 3. | MCC | 1-15% |
| 4. | Water | — |

Lubrication

| | | |
|---|---|---|
| 5. | Magnesium Stearate | 1-4% |

### Procedure:

Weighed and mixed the defined quantities of the sieved active ingredient & MCC. The blend was granulated using a solution of HPMC in water and the wet mass was passed through an extruder. The extruded mass was processed in an spheronizer to obtain pellets. The obtained pellets were dried.

The pellets remain uncoated or are film coated for immediate release of the drug. Immediate release pellets are then coated with rate control polymer coating solution for controlled release of the drug.

The film coating solution used to coat the pellets can either be of table 2 or 6. The controlled release coating solution used to coat the pellets for drug release at pH 5 to 6 of the gastro intestinal tract was either of table 3 or 8. The controlled release coating solution used to coat the pellets for drug release at pH 6 or 8 of the gastro intestinal tract was either of table 4 or 10.

The immediate release and controlled release pellets were filled inside a capsule; wherein the weight of the immediate release and controlled release pellets were present in the different proportion to provide an overall 24 hours drug effect of the dosage form.

### Dissolution of Controlled Release Capsule Containing Pellets:

The dissolution profile of the formulation of the present invention is as below:

TABLE 13
Dissolution Profile:

| | Time | | |
|---|---|---|---|
| Sr. No. | (Hours) | Dissolution Medium | Limits |
| 1 | 3 | 0.1 N HCl | 5%-25% |
| 2 | 6 | Trisodium buffer pH 5.5 | 25%-60% |
| 3 | 9 | Trisodium buffer pH 6.8 | 40%-70% |
| 4 | 12 | Trisodium buffer pH 6.8 | 50%-80% |
| 5 | 18 | Trisodium buffer pH 6.8 | NLT 70% |

1. Once a day controlled release solid oral formulation of anti-epileptic drugs in the form of capsule filled with immediate release and controlled release tablets; of which:
A) Immediate release tablets provide quick drug action and comprises of:
i) Tablet core of active pharmaceutical ingredient and pharmaceutical acceptable excipients.
Optionally coated with,
ii) Film coating comprising of film forming polymers and pharmaceutically acceptable excipients.
B) Controlled release tablets which release drug at pH 5 to 6 of gastrointestinal tract comprising:
i) Tablet core of active pharmaceutical ingredient and pharmaceutically acceptable excipients.
ii) Film coating comprising film forming polymer and pharmaceutically acceptable excipients; and
iii) Control release coating comprising control release polymer suitable to release the drug at pH 5 to 6 of gastrointestinal tract and pharmaceutically acceptable excipients.
C) Controlled release tablets which release drug at pH 6 to 8 of gastrointestinal tract comprising:
i) Tablet core of active pharmaceutical ingredient and pharmaceutically acceptable excipients.
ii) Film coating comprising film forming polymer, and pharmaceutically acceptable excipients; and
iii) Control release coating comprising control release polymers suitable to release the drug at pH 6 to 8 of gastro-intestinal tract and pharmaceutically acceptable excipients.

2. Once a day controlled release solid oral formulation as claimed in claim 1, wherein the antiepileptic drug is carbamazepine derivative preferably oxcarbazepine.

3. Once a day controlled release solid oral formulation as claimed in claim 1, wherein the tablet contained inside the capsule is 5 to 50% of the weight of the formulation.

4. Once a day controlled release solid oral formulation as claimed in claim 1, wherein the tablet core comprises of 10-90% active pharmaceutical agent, 1-15% diluent, 1-15% binder, 1-15% disintegrant and 0.25 to 10% lubricant of the weight of the core.

5. Once a day controlled release solid oral formulation as claimed in claim 1, wherein the amount of film coating is 0.5 to 5% weight gain over core weight and comprises of 1-20% coating polymer, 0.2-15% acid neutralizer, 0.1-15% plasticizer and 0.2-15% anti-settling agent.

6. Once a day controlled release solid oral formulation as claimed in claim 1, wherein the amount of controlled release coating solution used to coat the tablets releasing drug at pH 5 to 6 is 5 to 25% weight gain over the film coated immediate release tablets and amount of controlled release coating solution used to coat the tablets releasing drug at pH 6 to 8 is 5 to 50% weight gain over the film-coated immediate release tablets.

7. Once a day controlled release solid oral formulation as claimed in claim 1, wherein the controlled release coating solution comprises 2-95% of release controlling polymer, 0.1-20% of plasticizer, 0.01-15% of opacifier, 0.01-15% of emulsifier, 0.05-15% of antisettling agents and 0.02-15% of pigments.

8. Once a day controlled release solid oral formulation as claimed in claim 1, wherein the core of immediate release and controlled release tablets comprises of binder selected from the group comprising of starch, cellulose and cellulose derivatives, cross linked carboxy methyl cellulose, lactose, mannitol, polyethylene glycol, polyvinylpyrollidone, ethyl cellulose, polyvinyl alcohol and mixture thereof; diluent selected from the group comprising of starch, micro-crystalline cellulose, lactose, glucose, mannitol, alginates, alkali earth metal salts, clays, polyethylene glycol and mixture thereof; lubricant selected from the group comprising of magnesium stearate, other alkali earth metal stearate; colloidal anhydrous silica, talc, sodium stearyl fumarate, glyceryl monostearate, lauryl sulphate, hydrogenated vegetable oil, sodium benzoate, glyceryl mono stearate, poly ethylene glycol and mixture thereof; disintegrant selected from the group comprising of cross-linked polymers such as crospovidone, starch or modified starch such as sodium starch glycolate, clays such as bentonite or veegum, celluloses or cellulose derivatives, crosslinked cellulose such as croscarmellose sodium, resins such as polacrillin potassium, alginates, gums like xanthun gum, effervescent agent such as sodium bicarbonate and citric acid and mixture thereof.

9. Once a day controlled release solid oral formulation as claimed in claim 1, wherein the film forming polymer used in the film coating is selected from the group comprising of but not limited to zein, cellulose and cellulose derivatives like hydroxypropylmethylcellulose, hydroxyethyl cellulose, methyl cellulose, hydroxypropylcellulose; polyvinylpyrollidone, polyvinyl alcohol and mixture thereof.

10. Once a day controlled release solid oral formulation as claimed in claim 1, wherein controlled release coating polymers are selected from the group comprising of but not limited to methacrylic acid copolymers preferably methacrylic acid copolymer type-A, methacrylic acid copolymer type-B, methacrylic acid copolymer type-C, polyvinyl acetate phthalate, hydroxypropylmethyl-cellulose phthalate, cellulose acetate phthalate and mixture thereof.

11. Once a day controlled release solid oral formulation as claimed in claim 1, wherein the acid neutralizer is selected from the group comprising of magnesium oxide sodium hydroxide, potassium hydroxide, ammonia solution, ammonium chloride and mixture thereof; plasticizer is selected from the group comprising of polyethylene glycol, propylene glycol, diethyl phthalate, triethyl citrate, acetylated triethyl citrate, methyl citrate, triacetin and mixture thereof; anti settling agents is selected from the group comprising of talc, anhydrous silica sodium stearyl fumarate, glyceryl monostearate, lauryl sulphate, hydrogenated vegetable oil, sodium benzoate, glyceryl mono stearate, poly ethylene glycol and mixture thereof; pigments is selected from the group comprising of iron oxide, titanium dioxide, phthalocyanine blue and mixture thereof; emulsifier is selected, from the group comprising of tween-80, lecithin polysorbate, polyoxyethylene hardened castor oil, macrogol and mixture thereof.

12. Once a day controlled release solid oral formulation as claimed in claim 1, which is prepared by filling immediate and controlled release tablets inside capsule wherein the immediate releases tablets are prepared by compressing the granules into tablet, optionally coated with film coating and control release tablets are prepared by coating film coated tablets with rate control coating such that drug is released at varied pH environment of the gastrointestinal tract.

13. Once a day controlled release solid oral formulation of oxcarbazepine comprising immediate release and controlled release tablets filled inside capsule of which:
A) Immediate release tablets are uncoated or film coated and comprises:
i) Tablet core comprising 85.23% of active pharmaceutical ingredient, 5.68% of disintegrant, 2.87% of binder, 4.54% disintegrant in extra-granular part and 1.7% of lubricant along with suitable solvent.
ii) Film coating comprising 1.4% film coating polymer, 0.14% platicizer, 0.41% anti settling agent along with suitable solvent.
B) Controlled release tablets releasing drug at pH 5 to 6 of gastro-intestinal tract comprising:
i) Tablet core comprising 88.24% of active pharmaceutical ingredient 7.0% of disintegrant, 2.94% of binder and 1.82% of lubricant along with suitable solvent.
ii) Film coating comprising 1.4% of film, coating polymer, 0.14% of platicizer, 0.41% of anti settling agent along with suitable solvent.
iii) Control release coating solution comprising 9.29% of control release polymer, 0.27% of plasticizer and 0.44% of pigment along with suitable solvent.
C) Controlled release tablets releasing drug at pH 6 to 8 of gastro-intestinal tract comprising:
i) Tablet core comprising 86.20% of active pharmaceutical ingredient 12.07% of binder and 1.72% of lubricant along with suitable solvent.
ii) Film coating comprising 1.4% of film coating polymer, 0.14% of platicizer, 0.41% of anti settling agent along with suitable solvent.
iii) Control release coating solution comprising 12.06% control release polymer, 6.63% of acid neutralizer 2.98% of platicizer, 1.62% of pigment and 3.45% of anti-settling agent along with suitable solvent.

14. Once a day controlled release solid oral formulation as claimed in claim 1 wherein the dissolution of the dosage form occurs in following fashion:
i) At 3 hours in 0.1 N HCl dissolution medium 5-25%.
ii) At 6 hours in Trisodium buffer pH 5.5 medium 25-60%.
iii) At 9 hours in Trisodium buffer pH 6.8 medium 40-70%.
iv) At 12 hours in Trisodium buffer pH 6.8 medium 50-80%.
v) At 18 hours in Trisodium buffer pH 6.8 medium not less than 70%.

15. Once a day controlled release solid oral formulation as claimed in claim 13 wherein the dissolution of the dosage form occurs in following fashion:
i) At 3 hours in 0.1 N HCl dissolution medium 5-25%.
ii) At 6 hours in Trisodium buffer pH 15.5 medium 25-60%.
iii) At 9 hours in Trisodium buffer pH 6.8 medium 40-70%.
iv) At 12 hours in Trisodium buffer pH 6.8 medium 50-80%.
v) At 18 hours in Trisodium buffer pH 6.8 medium not less than 70%.
